(19) United States
(12) Patent Application Publication
(10) Pub. No.: US 2010/0249922 A1
(43) Pub. Date: Sep. 30, 2010

(54) FIBER-REINFORCED SYNTHETIC SHEETS FOR PROSTHETIC HEART VALVE LEAFLETS

(75) Inventors: XueMei Li, Shoreview, MN (US); Yi-Ren Woo, Livermore, CA (US)

Correspondence Address: Thomas A. Rendos;St Jude Medical Cardiovasuclar Division, 177 East County Road B, St Paul, MN, 55117, US

(73) Assignee: St Jude Medical Inc.

(21) Appl. No.: 12/733,762

(22) PCT Filed: Sep. 19, 2008
(86) PCT No.: PCT/US08/10889

§ 371 Date: Mar. 18, 2010

Related U.S. Application Data
(60) Provisional application No. 60/994,527, filed on Sep. 19, 2007.

(51) Int. Cl. A61F 2/24

(52) U.S. Cl. 623/2.17; 623/2.12

(57) ABSTRACT

A structure for use as a prosthetic heart valve leaflet includes a two-dimensional array that is made up of one or more fiber strands and that is covered by a web of polymer material to produce a sheet that is impervious to blood. This sheet is stretchable along two axes that lie in the plane of the sheet and that are at an angle to one another. The sheet may be more easily stretched along one of these axes than along the other axis. Along each axis the amount of force required to produce initial increments of stretch may be less than the amount of force required to produce subsequent increments of stretch (i.e., increments of stretch beyond the initial increments).

This application claims the benefit of U.S. provisional patent application No. 60/994,527, filed Sep. 19, 2007, which is hereby incorporated by reference herein in its entirety.

### BACKGROUND OF THE INVENTION

The natural heart valve leaflet has anisotropic mechanical properties and is more extensible in the radial direction than in the circumferential direction. The “radial direction” in a heart valve leaflet is the direction that extends from the base of the leaflet to the free edge of the leaflet. The “circumferential direction” in a heart valve leaflet is a direction that is generally annular of the heart valve (like the native annulus of the heart valve is annular of the heart valve). The “circumferential direction” extends along a heart valve leaflet from one commissure of the heart valve to another circumferentially adjacent commissure of the heart valve.

When a natural heart valve is diseased or otherwise damaged, replacement can become necessary. Replacement (prosthetic) heart valves can be constructed using a flexible elastomer for the leaflets. When used for construction of heart valve leaflets, elastomers, due to lack of mechanical strength, are often reinforced by synthetic fibers (see, for example, U.S. Pat. Nos. 4,192,020; 4,340,091; 4,731,074; 6,726,715; and 6,916,338). However, synthetic fibers are much less extensible than natural leaflet tissue, and even less extensible than chemically fixed leaflet tissue (which is another material that is sometimes used for prosthetic heart valve leaflets). To mimic the extensibility and anisotropic properties of natural leaflets, approaches such as different fiber density arrangements throughout the synthetic leaflet and processes to crimp woven fabric to increase its extensibility have been proposed. These approaches can be difficult to implement and/or may not be able to achieve desired properties.

### SUMMARY OF THE INVENTION

In accordance with certain possible aspects of the invention, a prosthetic heart valve leaflet may include (1) a perforate, flexible, two-dimensional array that is formed from at least one fiber strand and that can be handled (at least to some degree) without coming apart, and (2) a web of flexible polymer material secured to and covering the array so that the combination of the array and the web is impervious to blood flow. The array is stretchable along each of first and second axes that lie in the two dimensions of the array and that are perpendicular or at an angle to one another. The web is stretchable with the array along each of the first and second axes.

The array may be stretchable along each of the first and second axes in successive first and second phases. In the first phase the stretchability may be due primarily to deformation of a pattern of the strand(s) in the array and without significant elongation of the strand(s). In the second phase, the stretchability may be due, at least in part, to elongation of the strand(s).

In cases having the above-mentioned first and second phases, less force may be required to produce a unit of stretch in the first phase than is required to produce a unit of stretch in the second phase.

In accordance with another possible aspect of the invention, in cases having the above-mentioned first and second phases, less force may be required to produce a given amount of stretch along the first axis in the first phase than is required to produce that amount of stretch along the second axis in the first phase. In addition, less force may be required to produce a unit of stretch along the first axis in the second phase than is required to produce a unit of stretch along the second axis in the second phase. Still further, for stretch along each of the first and second axes, less force may be required to produce a unit of stretch in the first phase than is required to produce a unit of stretch in the second phase.

The leaflet may be used in a prosthetic heart valve that includes a structure for supporting the leaflet between first and second commissure portions (e.g., posts) of the structure. In such a case, the leaflet may be oriented so that the second axis extends between the first and second commissure portions. Assuming such orientation, the array may be formed so that less force is required to produce a given amount of stretch along the first axis than is required to produce that same amount of stretch along the second axis. In such a case, and along each of the first and second axes, the stretchability of the array may include successive first and second phases. The force required to produce a unit of stretch in the first phase may be less than the force required to produce a unit of stretch in the second phase.

The above-mentioned array may be formed by knitting the fiber strand(s). Alternatively, the above-mentioned array may be formed by weaving the fiber strand(s).

Examples of materials that may be used for the fiber strand(s) include polyester, polyethylene, polytetrafluoroethylene, polypropylene, nylon, etc.

The array may be embedded in the material of the web. Examples of materials that may be used for the web include polyurethane, silicone rubber, fluoroelastomer, SIBS (poly(stryene-b-isobutylene-b-styrne)), PVA (polyvinyl alcohol) hydrogel, etc.

Further features of the invention, its nature and various advantages, will be more apparent from the accompanying drawings and the following detailed description.

### BRIEF DESCRIPTION OF THE DRAWINGS

FIG. 1 is a simplified depiction of an illustrative embodiment of one component of prosthetic heart valve leaflet structure in accordance with the invention.

FIG. 2 is a view similar to FIG. 1, but shows the FIG. 1 component in another operating condition.

FIG. 3 is a simplified cross sectional view of an illustrative embodiment of a prosthetic heart valve leaflet structure in accordance with the invention.

FIG. 4 is a simplified diagram showing illustrative operating characteristics of a prosthetic heart valve leaflet structure in accordance with the invention.

FIG. 5 is a simplified perspective or isometric view of an illustrative embodiment of a prosthetic heart valve in accordance with the invention.

FIG. 6 shows again what is shown in FIG. 4 with some additional parameters indicated.

### DETAILED DESCRIPTION

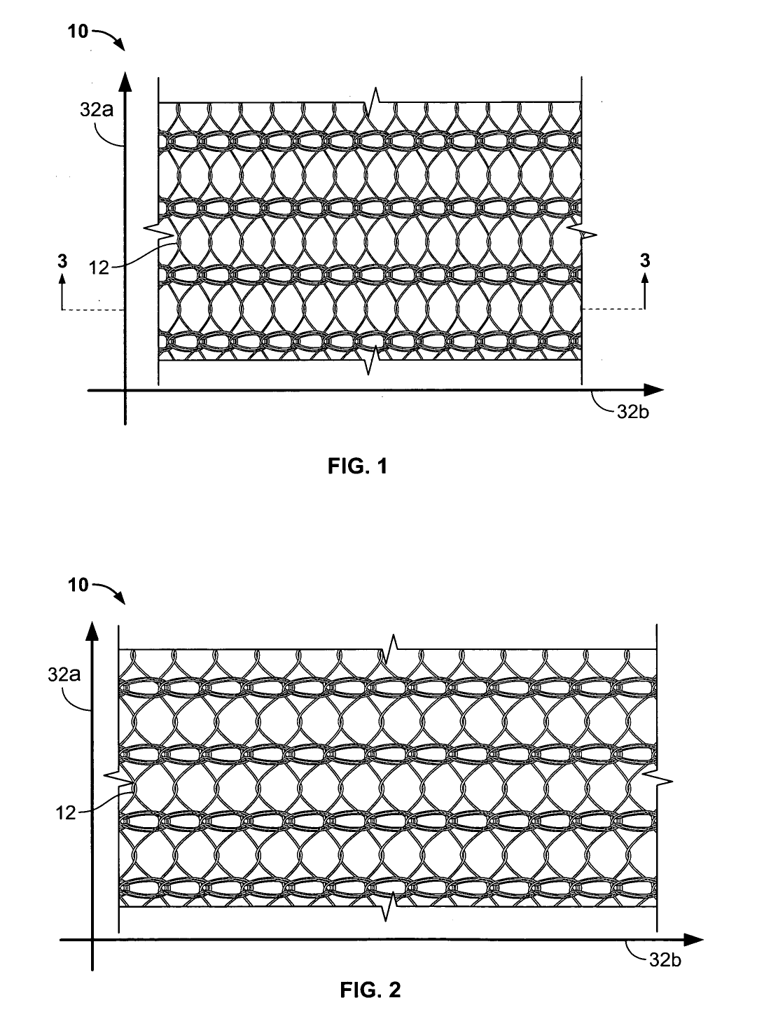

A structural design of fabric can be used to mimic the extensible and anisotropic properties of natural tissue. Solely synthetic fiber, in the direction of its length, might not be extensible enough to perform the leaflet function of a heart valve. When the fiber bundle is arranged to form a mesh-like structure that has lower elastic modulus (its stress per unit strain) when bearing relatively low loads and a higher modulus when bearing greater loads, it can mimic the mechanical property of the natural heart valve leaflet tissue. As an example, FIG. 1 shows the structural arrangement of the fiber bundle in a fabric 10 in its natural (unstressed) state. Fabric 10 is formed from at least one fiber strand 12. The way in which structure 10 has been formed from strand material 12 allows a reasonable amount of fabric stretch and flexibility as shown in FIG. 2 when the structure takes a lower load and before the material property of the fiber bundle 12 starts to play a major role.

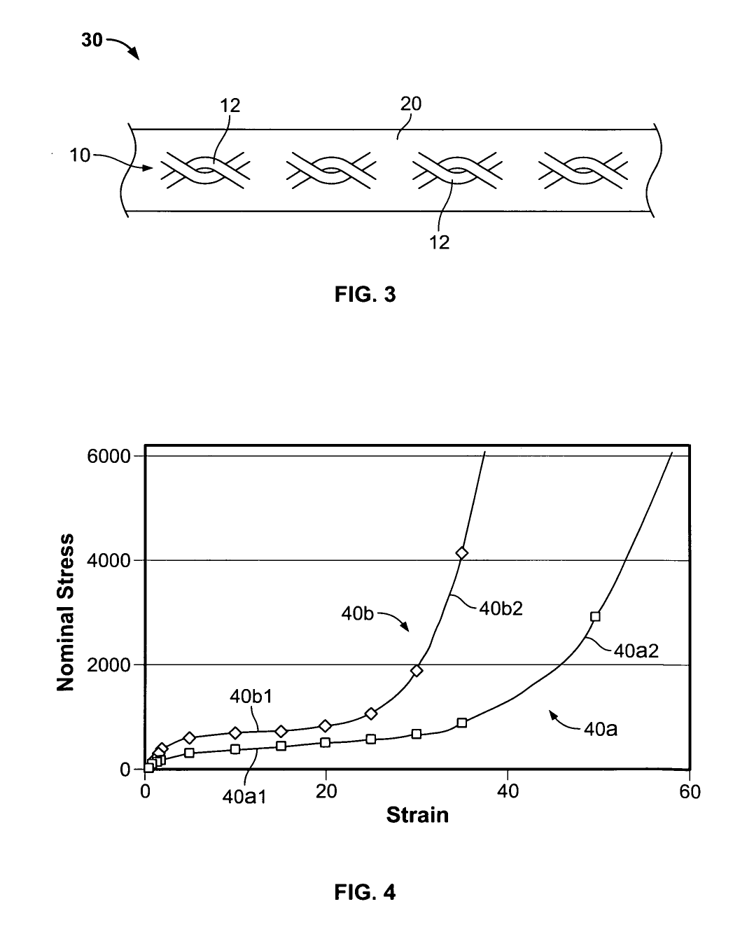

FIG. 3 shows how fabric 10 can be embedded in a web 20 of elastomeric material to produce a sheet of prosthetic heart valve leaflet material 30. (FIG. 3 is a simplified cross sectional view of such a sheet taken as indicated by the line 3-3 in FIG. 1.) The resulting structure 30 is, of course, impervious to blood. Both components 10 and 20 stretch together in response to any force applied to structure 30.

FIG. 4 shows illustrative stress-strain curves for structure 30. The stress-strain curves in FIG. 4 show a very similar mechanical behavior to that of natural leaflet tissue, namely, a long-and-low-load-bearing toe area toward the left in FIG. 4 that is achieved by the deformation of the structure of fabric 10 (e.g., as it goes from an unstretched condition like that shown in FIG. 1 to a stretched condition like that shown in FIG. 2), and then a rapidly increasing load-bearing area toward the right in FIG. 4 that mainly results from the material property of the fibers 12 in fabric 10. Again, it is emphasized that the preceding sentence makes a distinction between (1) performance of structure 30 toward the left in FIG. 4 (where “structural” factors such as the pattern in which the fibers are assembled (e.g., knitted) to produce fabric 10 have greater effect on fabric behavior), and (2) performance of structure 30 toward the right in that FIG. (where “material property” factors such as the modulus (e.g., in tension and flexure) of individual fiber strands 12 begin to have greater effect on fabric behavior).

Fabric 10 can also be designed to be more extensible in the radial direction (lower and more right-ward curve 40a in FIG. 4) and less extensible in the circumferential direction (upper and more left-ward curve 40b in FIG. 4) for a given amount of stress. The phrases “radial direction” and “circumferential direction” are again used as defined in the Background section of this specification. In FIGS. 1 and 2 the radial direction is vertical (parallel to axis 32a) and the circumferential (commissure-to-commissure) direction is horizontal (parallel to axis 32b). Each of the curves in FIG. 4 is the result of a separate uniaxial stress test. The vertical axis in FIG. 4 is stress in kilo Pascals (KPa). The horizontal axis is percent strain.

The presently disclosed expedient of using structural design of the fiber bundle 10 as reinforcement of the matrix material 20 (e.g., silicone) of prosthetic heart valve leaflets 30 is much easier to implement than the above-mentioned prior approaches. Instead of developing special methods and machining for different density arrangements throughout the leaflets, textile methods, such as knitting, can readily be used for fabric 10 manufacturing. Impregnating or coating the matrix material 20 on the fabric 10 is also readily implemented in the manufactory.

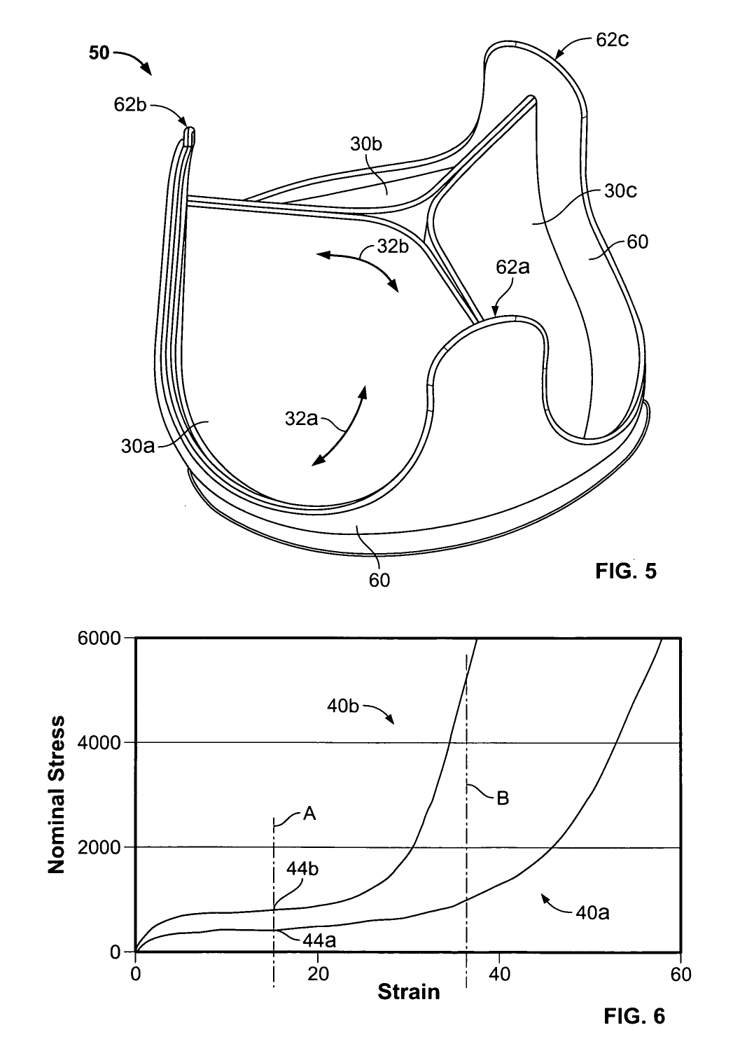

This invention can be applied to the production of prosthetic heart valves with synthetic leaflets. For example, FIG. 5 shows an illustrative embodiment of such a prosthetic heart valve 50. As shown in FIG. 5, heart valve 50 includes an annular supporting structure 60 having three circumferentially spaced commissure regions 62a-c. Three leaflets 30a-c (each made as described above) are mounted in supporting structure 50. In FIG. 5 leaflets 30a-c are shown as coming together along their upper free edges to close the valve to reverse blood flow (which would be in the downward direction as viewed in FIG. 5). When blood pressure below the valve is greater than blood pressure above the valve, the free edges of leaflets 30a-c can move apart to open the valve and allow blood to flow upwardly through it. Note, in particular, that the upper portion of leaflet 30a extends between circumferentially adjacent commissure regions 62a and 62b; the upper portion of leaflet 30b extends between circumferentially adjacent commissure regions 62b and 62c; and the upper portion of leaflet 30c extends between circumferentially adjacent commissure regions 62c and 62a. On representative leaflet 30a the “circumferential direction” (as that term is used elsewhere in this specification) is indicated by double-headed arrow 32b, and the “radial direction” (as that term is used elsewhere in this specification) is indicated by double-headed arrow 32a. Each of leaflets 30a-c is preferably mounted in supporting structure 60 so that the axis along which it has properties like those shown by curve 40a in FIG. 4 is parallel to radial direction 32a, and so that the axis along which it has properties like those shown by curve 40b in FIG. 4 is parallel to circumferential direction 32b. In this way the behavior of each leaflet 30a-c can mimic the behavior of native heart valve leaflets (including the anistropic behavior of such native leaflets).

Recapitulating and amplifying the above, the term “fabric” as used herein refers to a two-dimensional array 10 of fibers or fiber segments 12 that is, in the absence of external forces, able to retain by itself (i.e., even prior to the addition of the matrix material 20 that is used to convert it to heart valve leaflet material 30) the arrangement of fibers or fiber segments it is given as it is made. In this way the fabric 10 can be made on one piece of machinery and then easily moved to other machinery for addition of the matrix material 20 without disturbing the pattern of fibers or fiber segments 12 given to the fabric by the first machinery. In other words, the array 10 of fibers 12 formed by the first machinery can be handled without coming apart in order, for example, to move the array to other machinery for further processing. Examples of suitable materials for use as the fibers 12 of the fabric 10 are polyester, polyethylene, PTFE, polypropylene, nylon, etc. Examples of suitable matrix materials 20 for subsequent addition to the fabric 10 are polyurethane, silicone rubber, fluoroelastomer, SIBS, PVA hydrogel, etc. The elastomer 20 is typically applied to produce a fabric-and-elastomer composite 30 that is a blood-impervious sheet or web suitable for use as prosthetic heart valve leaflet material.

The fabric 10 is “engineered” to give the composite 30 of the fabric 10 and the matrix material 20 certain preferred properties. This means selecting the fiber strand(s) 12 to be used in making the fabric 10 and selecting the pattern that will be used for assembling (forming) the fiber strand(s) into the fabric. However, the fabric 10 is basically two-dimensional (except for thickness of the fiber strand(s) 12 and extra thickness that results from the strand material crossing over or under other strand material). The invention preferably does not rely on or employ deformation of the fabric or portions of the fabric 10 into a third dimension (i.e., out of the plane of the paper on which FIGS. 1 and 2 are drawn) to give the fabric or the resulting leaflet 30 desired stress/strain properties. For example, the invention preferably does not rely on or employ crimping of the fabric or portions of the fabric into such a third dimension. The fabric 10 may be either custom-made for use in accordance with the invention, or it may be possible to select already available fabric that is suitable. The following may be among the preferred properties of the composite 30. The composite 30 preferably has an elastic modulus that is relatively low at relatively low levels of stress (e.g., at 40a1 or 40b1 in FIG. 4), and that becomes higher at higher levels of stress (e.g., at 40a2 or 40b2 in FIG. 4). In especially preferred embodiments, extensibility of the composite material 30 is anistropic. In particular, the extensibility or stretchability is greater in what will be the radial direction 32a when the material 30 is used as a prosthetic heart valve leaflet (e.g., as in curve 40a in FIG. 4), and it is less extensible or stretchable in what will be the circumferential direction 32b of the heart valve leaflet (e.g., as in curve 40b in FIG. 4).

Knitting is an especially preferred technique for making the fabric 10. Another example of fabric-making techniques that can be used is weaving.

Reviewing portions of the foregoing in terms that may, to some extent, be different from those used at certain other points in this specification, a prosthetic heart valve leaflet 30 may include a perforate, flexible, two-dimensional array 10 that is formed from at least one fiber strand 12. This array 10 can be handled (at least to some extent) without coming apart. The array 10 is stretchable (strain in FIG. 4) along each of first and second axes (e.g., 32a and 32b in FIGS. 1 and 2) that lie in the two dimensions of the array (e.g., the plane of the paper on which FIGS. 1 and 2 are drawn) and that are perpendicular or at an angle to one another. The leaflets 30 may further include a web 20 of flexible polymer material secured to and covering the array 10 so that the combination of the array and the web is impervious to blood flow. The web 20 is stretchable (strain in FIG. 4) with the array 10 along each of the first and second axes 32a and 32b.

The array 10 may be stretchable (strain in FIG. 4) in successive first and second phases (e.g., successive curve portions 40a1 and 40a2 in the case of curve 40a, or successive curve portions 40b1 and 40b2 in the case of curve 40b in FIG. 4). The first phase 40a1 or 40b1 of stretchability may be due primarily to deformation of a fiber pattern in the array (e.g., change in the pattern of fiber(s) 12 from FIG. 1 to FIG. 2). The second phase 40a2 or 40b2 of stretchability may be due primarily to elongation of the fiber strand(s) 12 in array 10.

Less force (nominal stress in FIG. 4) may be required to produce a unit of stretch (strain in FIG. 4) in the above-mentioned first phase 40a1 or 40b1 than is required to produce a unit of stretch in the above-mentioned second phase 40a2 or 40b2. This means, for example, that the upward slope of curve 40a in FIG. 4 is not as steep in first phase 40a1 as it is in second phase 40a2. The same is true for curve 40b (i.e., first phase 40b1 is less steep than second phase 40b2).

Less force (nominal stress in FIG. 4) may be required to produce a given amount of stretch (strain in FIG. 4) along the first axis 32a in the first phase 40a1 than is required to produce that amount of stretch along the second axis 32b in the first phase 40b1. This is illustrated by FIG. 6 (which reproduces the data from FIG. 4 so that other parameters can be indicated without unduly complicating FIG. 4). For example, FIG. 6 shows that to produce 15% strain (i.e., at line A), less force 44a is required along axis 32a (behavior curve 40a) than is required (at 44b) to produce the same amount of strain along axis 32b (behavior curve 40b).

Less force (nominal stress in FIG. 4) may be required to produce a unit of stretch (strain in FIG. 4) along the first axis 32a in the second phase 40a2 than is required to produce a unit of stretch along the second axis 32b in the second phase 40b2. This means, for example, that the upward slope of curve portion 40a2 is less steep than the upward slope of curve portion 40b2. Also in such a case, for stretch along each of the first and second axes, less force may be required to produce a unit of stretch in the first phase 40a1 or 40b1 than is required to produce a unit of stretch in the second phase 40a2 or 40b2. This means, for example, that the upward slope of curve portion 40a1 is not as steep as the upward slope of curve portion 40a2 (and similarly for curve portions 40b1 and 40b2).

A prosthetic heart valve 50 may include the leaflet structure 30 described earlier (e.g., as at 30a in FIG. 5) and a structure 60 for supporting the leaflet between first and second commissure portions (e.g., 62a and 62b) of the supporting structure.

In such a valve 50 the leaflet (e.g., 30a) may be oriented so that the second axis 32b extends between the first and second commissure portions (e.g., 62a and 62b). In such a case, the array 10 may be formed so that less force (nominal stress) is required to produce a given amount of stretch (strain) along the first axis 32a than is required to produce that amount of stretch along the second axis 32b. This is illustrated, for example, by FIG. 6 in which the force required to produce amount of strain A is less along curve 40a than along curve 40b. The same is true for other amounts of strain (e.g., amount of strain B in FIG. 6, which takes both of curves 40a and 40b into their second, steeper phases 40a2 and 40b2). Still further in such a case, along each of the first and second axes 32a and 32b the stretchability of the array 10 may include successive first and second phases (e.g., successive curve portions 40a1 and 40a2, or successive curve portions 40b1 and 40b2). The force (nominal stress) required to produce a unit of stretch (strain) in the first phase 40a1 or 40b1 may be less than the force required to produce a unit of stretch in the second phase 40a2 or 40b2. This is illustrated, for example, by curve portion 40a1 being less steep than curve portion 40a2 (and similarly for curve portions 40b1 and 40b2).

The leaflet 30 may include an array 10 that is formed by knitting fiber strand(s) 12. Alternatively, array 10 may be formed by weaving fiber strand(s) 12. The array 10 may be embedded in the material of web 20.

It will be understood that the foregoing is only illustrative of the principles of the invention, and that various modifications can be made by those skilled in the art without departing from the scope and spirit of the invention. For example, the particular valve shape and configuration shown in FIG. 5 is only illustrative, and the invention is equally applicable to prosthetic heart valves having other shapes and/or configurations.

The invention claimed is:

1. A prosthetic heart valve leaflet comprising:
a perforate, flexible, two-dimensional array that is formed from at least one fiber strand and that can be handled without coming apart, the array being stretchable along each of first and second axes that lie in the two dimensions of the array and that are at an angle to one another; and
a web of flexible polymer material secured to and covering the array so that the combination of the array and the web is impervious to blood flow, the web being stretchable with the array along each of the first and second axes, and wherein along each of the axes the amount of force required to produce initial increments of stretch of the leaflet is less than the amount of force required to produce subsequent increments of stretch of the leaflet.

2. The leaflet defined in claim 1 wherein the array is stretchable along each of the first and second axes in successive first and second phases, wherein in the first phase the stretchability is due to deformation of a fiber pattern in the array without significant elongation of the least one fiber strand, and wherein in the second phase the stretchability is due, at least in part, to elongation of the at least one fiber strand.

3. The leaflet defined in claim 2 wherein less force is required to produce a unit of stretch in the first phase than is required to produce a unit of stretch in the second phase.

4. The leaflet defined in claim 2 wherein less force is required to produce a given amount of stretch along the first axis in the first phase than is required to produce that amount of stretch along the second axis in the first phase.

5. The leaflet defined in claim 4 wherein less force is required to produce a unit of stretch along the first axis in the second phase than is required to produce a unit of stretch along the second axis in the second phase.

6. The leaflet defined in claim 5 wherein, for stretch along each of the first and second axes, less force is required to produce a unit of stretch in the first phase than is required to produce a unit of stretch in the second phase.

7. A prosthetic heart valve comprising:
the leaflet defined in claim 1; and
a structure for supporting the leaflet between first and second commissure portions of the structure.

8. The valve defined in claim 7 wherein the leaflet is oriented so that the second axis extends between the first and second commissure portions.

9. The leaflet defined in claim 1 wherein the array is formed by knitting the at least one fiber strand.

10. The leaflet defined in claim 1 wherein the array is formed by weaving the at least one fiber strand.

11. The leaflet defined in claim 1 wherein the at least one fiber strand is made of a material selected from the group consisting of polyester, polyethylene, polytetrafluoroethylene, polypropylene, and nylon.

12. The leaflet defined in claim 1 wherein the array is embedded in the material of the web.

13. The leaflet defined in claim 1 wherein the material of the web is selected from the group consisting of polyurethane, silicone rubber, fluoroelastomer, SIBS, and PVA hydrogel.

14. The valve defined in claim 8 wherein the array is formed so that less force is required to produce a given amount of stretch along the first axis than is required to produce that amount of stretch along the second axis.

15. The valve defined in claim 14 wherein, along each of the first and second axes, the stretchability of the array includes successive first and second phases, the force required to produce a unit of stretch in the first phase being less than the force required to produce a unit of stretch in the second phase.
